(19) United States
(12) Patent Application Publication
(10) Pub. No.: US 2009/0176216 A1
(43) Pub. Date: Jul. 9, 2009

(54) Alleles and polymorphisms predictive of responsiveness to biologic therapy in psoriasis

(76) Inventor: Wayne Gulliver, St. John's (CA)

Correspondence Address: LAHIVE & COCKFIELD, LLP;FLOOR 30, SUITE 3000, ONE POST OFFICE SQUARE, BOSTON, MA, 02109, US

(21) Appl. No.: 12/082,811

(22) Filed: Apr. 14, 2008

Related U.S. Application Data
(60) Provisional application No. 60/923,350, filed on Apr. 13, 2007.

(51) Int. Cl. C12Q 1/68; G06F 17/30

(52) U.S. Cl. 435/6; 707/104.1; 707/5

(57) ABSTRACT

The invention provides methods for predicting responsiveness to a biologic agent in a subject suffering from psoriasis. The methods involve assaying for the presence or absence of an allele or polymorphism in the subject that is predictive of responsiveness to biologic therapy, such as an HLA-Cw6 allele, a TNFα 238 polymorphism or a TNFα 308 polymorphism. The methods can further comprise selecting a treatment regimen with a biologic agent in a psoriasis subject based upon presence or absence of the allele or polymorphism in the subject. The methods can further comprise administering a biologic agent to the subject according to the selected treatment regimen. Kits that include means for assaying an allele or polymorphism that is predictive of responsiveness to biologic therapy for psoriasis are also provided. Methods of preparing and using databases, and computer program products therefore, for selecting a psoriasis subject for treatment with a biologic agent are also provided.

### RELATED APPLICATION

This application claims priority to U.S. Provisional Application No. 60/923,350, Attorney Docket No. NCRI-001-1, filed Apr. 13, 2007, entitled “Alleles and Polymorphisms Predictive of Responsiveness to Biologic Therapy in Psoriasis.” The contents of any patents, patent applications, and references cited throughout this specification are hereby incorporated by reference in their entireties.

### BACKGROUND OF THE INVENTION

Psoriasis is a common inherited inflammatory dermatosis with a population prevalence of approximately 2-3% in North America and most European countries (Ahmeen, M. (2003) Pharmacogenomics 4:297-308). Psoriasis often has an early onset, between 10 and 30 years of age, and is a chronic, life-long disease. It can cause reduction in physical and mental function and have a significant impact on quality of life. Physically, psoriasis is characterized by epidermal keratinocyte hyperproliferation, altered differentiation, vascular proliferation, inflammatory cell accumulation and leukocyte infiltration of primarily activated lymphocytes and neutrophils, with a phenotypic expression of well circumscribed erythematous scaling plaques scattered in a symmetrical distribution across areas of affected skin (Camp, R. D. R Psoriasis in Textbook of Dermatology, Champion, R. H. et al. (eds), Oxford:Blackwell Sciences, 1998, pp. 1589-1649; Park, B. S. et al. (1999) J. Invest. Dermatol. 112:113-116; Balendran, N. et al. (1999) J. Invest. Dermatol. 113:322-328). Furthermore, the disease can be complicated by severe arthritis (Park, B. S. et al. (1999) supra), referred to as psoriatic arthritis (PsA). The emotional distress of the disease can include frustration with ineffective treatment, fear of exacerbation of the disease, embarrassment and depression.

Traditional treatments for the management of mild-to-moderate psoriasis have included topical treatments such as corticosteroids, dithranol (anthralin derivative), coal tar and vitamin D₃ analogues, as well as phototherapy (Ahmeen, M. (2003) Pharmacogenomics 4:297-308). More severe disease requires systemic immunosuppressive therapy, often for long periods. Commonly used immunosuppressants include cyclosporine and methotrexate, although each can be associated with adverse toxicities (Ahmeen, M. (2003) supra). Furthermore, phototherapy using ultraviolet A with psoralen (PVA) may increase risk of skin cancers, including the possibility of malignant melanoma (Peritz, A. E. et al. (1999) J. Invest. Dermatol. Symp. Proc. 4:11-16). Accordingly, additional therapies for psoriasis have been sought.

More recently, biological therapies have been applied to the treatment of psoriasis. For example, RAPTIVA™ (efalizumab), a humanized anti-CD11a monoclonal antibody (mAb), and AMEVIVE™ (alefacept), an LFA3-Ig Fc fusion protein, both have been approved by the Federal Drug Administration (FDA) for the treatment of moderate to severe chronic plaque psoriasis. Additionally, three TNFα inhibitors, REMICADE™ (infliximab), a chimeric anti-TNFα mAb, ENBREL™ (etanercept), a TNFR-Ig Fc fusion protein, and HUMIRA™ (adalimumab), a human anti-TNFα mAb, have been approved by the FDA for treatment of psoriatic arthritis. While such biologic therapies have demonstrated some success in the treatment of psoriasis, not all subjects treated respond to biologic therapy. The use of biologic agents typically is more expensive than traditional treatments and usually requires administration by injection, which may require that the patient visit a medical office on a frequent basis. Thus, it would be very helpful to predict in advance of treatment whether a psoriasis patient is likely to be responsive to treatment with a biologic agent. Accordingly, ways for predicting responsiveness to biologic therapy in psoriasis patients are of particular interest.

### SUMMARY OF THE INVENTION

This invention provides methods and compositions for predicting responsiveness to a biologic agent in a subject having psoriasis based on the discovery that particular alleles or polymorphisms in the subject correlate with responsiveness to biologic therapy. Accordingly, in one aspect, the invention pertains to a method for predicting responsiveness to a biologic agent in a subject having psoriasis, wherein the method comprises assaying the subject for the presence or absence of an allele or polymorphism predictive of responsiveness to biologic therapy in psoriasis, and predicting responsiveness of the subject to the biologic agent based on the presence or absence of the allele or polymorphism in the subject. The subject can be assayed for the presence or absence of the allele or polymorphism by, for example, obtaining a genomic DNA sample from the subject and detecting the presence or absence of the allele or polymorphism in the genomic DNA sample.

In other embodiments, the method for predicting responsiveness to a biologic agent in a subject having psoriasis further comprises selecting a treatment regimen with the biologic agent based upon the presence or absence of the allele or polymorphism in the subject. Moreover, the method can further comprise administering the biologic agent to the subject according to the treatment regimen such that psoriasis is inhibited in the subject.

In one embodiment, the allele or polymorphism predictive of responsiveness to biologic therapy in psoriasis is an HLA-Cw6 allele. For example, presence of the HLA-Cw6 allele can be used to predict responsiveness of the subject to biologic therapy, whereas absence of the HLA-Cw6 allele can be used to predict lack of, or reduced, responsiveness of the subject to biologic therapy.

In other embodiments, the allele or polymorphism predictive of responsiveness to biologic therapy in psoriasis is a tumor necrosis factor alpha (TNFα) 238 polymorphism or a tumor necrosis factor alpha (TNFα) 308 polymorphism. Additional alleles or polymorphisms that may be predictive of responsiveness to biologic therapy in psoriasis are discussed further herein.

In one embodiment, the biologic agent is a TNFα inhibitor, non-limiting examples of which include etanercept, infliximab and adalimumab. In other embodiments, the biologic agent is an immune response modifier, non-limiting examples of which include LFA-3 modulators, such as alefacept, and CD11a modulators, such as efalizumab. Additional biological agents of potential use in the treatment of psoriasis are discussed further herein.

Preferably, the subject is a human subject. Preferably, psoriasis is chronic plaque psoriasis, such as moderate to severe chronic plaque psoriasis. In one embodiment, the subject suffers from chronic plaque psoriasis without psoriatic arthritis. In another embodiment, the subject suffers from chronic plaque psoriasis with psoriatic arthritis.

In another aspect, the invention pertains to kits for predicting responsiveness to a biologic agent in a subject having psoriasis. In one embodiment, the kit comprises:

a) means for detecting presence or absence in the subject of an allele or polymorphism predictive of responsiveness to biologic therapy in psoriasis; and

b) instructions for use of the kit to predicting responsiveness to a biologic agent in a subject having psoriasis.

For example, the means for detecting presence or absence in the subject of an allele or polymorphism predictive of responsiveness to biologic therapy in psoriasis can comprise a nucleic acid preparation (containing or more nucleic acid primers or probes) sufficient to detect presence or absence of the allele or polymorphism in a genomic DNA sample from the subject. In certain embodiments, the kit further comprises a biologic agent for treating psoriasis in the subject.

In one embodiment of the kit, the allele or polymorphism predictive of responsiveness to biologic therapy in psoriasis is an HLA-Cw6 allele. For example, the instructions within the kit can instruct that presence of the HLA-Cw6 allele can be used to predict responsiveness of the subject to biologic therapy. Alternatively, the instructions within the kit can instruct that absence of the HLA-Cw6 allele can be used to predict lack of, or reduced, responsiveness of the subject to biologic therapy.

In other embodiments, the allele or polymorphism predictive of responsiveness to biologic therapy in psoriasis is a tumor necrosis factor alpha (TNFα) 238 polymorphism or a tumor necrosis factor alpha (TNFα) 308 polymorphism. Additional alleles or polymorphisms that may be predictive of responsiveness to biologic therapy in psoriasis are discussed further herein.

In certain embodiments, the biologic agent incorporated into the kit is a TNFα inhibitor, non-limiting examples of which include etanercept, infliximab and adalimumab. In other embodiments, the biologic agent incorporated into the kit is an immune response modifier, non-limiting examples of which include LFA-3 modulator, such as alefacept, and CD11a modulators, such as efalizumab. Additional biological agents of potential use in the treatment of psoriasis that can be incorporated into the kit are discussed further herein.

Preferably, the kit is designed for use in a human subject, more preferably a human subject suffering from chronic plaque psoriasis, such as moderate to severe chronic plaque psoriasis. In one embodiment, the subject suffers from chronic plaque psoriasis without psoriatic arthritis. In another embodiment, the subject suffers from chronic plaque psoriasis with psoriatic arthritis.

Additional aspects of the invention pertain to a method of building a database, and computer program products useful for carrying out the method, for use in selecting a subject having psoriasis for treatment with a biologic agent. The method of building the database can comprise: receiving, in a computer system, genotypes, at one or more alleles or polymorphisms predictive of responsiveness to biologic agent treatment, from a plurality of subjects having psoriasis; and storing the genotype from each subject such that the genotype is associated with an identifier of the subject, such as a name of the subject or a numerical identifier coded to the identity of the subject. The method of building the database (and computer program products therefor) can further comprise receiving, in the computer system, one or more treatment regimens for treatment of psoriasis in a subject such that the treatment regimen is associated with the genotype of the subject and the identifier of the subject.

Additional aspects of the invention pertain to a method of selecting a psoriasis subject for a treatment with a biologic agent using a database, and computer program products useful for carrying out the method. The method of selecting the psoriasis subject for a treatment with a biologic agent can comprise: identifying, in a database comprising a plurality of psoriasis subjects, a subject whose database entry is associated with a genotype at one or more alleles or polymorphisms that is predictive of responsiveness to treatment with a biologic agent; and selecting the subject for treatment with a biologic agent. The method of selecting the psoriasis subject for a treatment with a biologic agent (and computer program products therefor) can further comprise selecting a treatment regimen by identifying, in the database, a treatment regimen that has been associated with the genotype of the subject and with an identifier of the subject.

### DETAILED DESCRIPTION OF THE INVENTION

This invention provides methods for predicting responsiveness to biologic therapy of a subject suffering from psoriasis, and methods for selecting a treatment regimen with a biologic agent, based on expression of particular alleles or polymorphisms in the subject to be treated. The invention is based, at least in part, on the observation that the presence of certain alleles or polymorphisms in the subject suffering from psoriasis is associated with increased or decreased responsiveness to therapy with biologic agents (see Example 1). In particular, certain alleles and polymorphisms that previously have been associated with increased likelihood of developing psoriasis now have been found to correlate with increased likelihood of responsiveness to biologic agent therapy. In contrast, the absence of such alleles or polymorphisms in a psoriasis subject has been found to correlate with a decreased likelihood of responsiveness to biologic agent therapy. Accordingly, the presence or absence of particular alleles or polymorphisms can be assessed in psoriasis subjects for which biologic agent therapy is being considered to thereby predict responsiveness of the subject to such therapy and/or to aid in the selection of an appropriate treatment regimen.

In order that the present invention may be more readily understood, certain terms are first defined. Additional definitions are set forth throughout the detailed description.

The term “predicting responsiveness to a biologic agent”, as used herein, is intended to refer to an ability to assess the likelihood that treatment of a subject with a biologic agent will or will not be effective in (e.g., provide a measurable benefit to) the subject. In particular, such an ability to assess the likelihood that treatment will or will not be effective typically is exercised before treatment with the biologic agent is begun in the subject. However, it is also possible that such an ability to assess the likelihood that treatment will or will not be effective can be exercised after treatment has begun but before an indicator of effectiveness (e.g., an indicator of measurable benefit) has been observed in the subject.

The term “allele”, as used herein, is intended to refer to one of a group of possible DNA codings (e.g., genes) occupying a particular position (locus) on a chromosome. An individual's “haplotype” is intended to refer to the particular allele that an individual happens to possess at that particular-position (locus) on the chromosome, whereas an individual's “genotype” is intended to refer to the set of alleles that the individual happens to possess at that particular position (locus). For example, for diploid organisms such as humans, an individual has two alleles, one on each chromosome, at each position (locus) that make up the individual's genotype, which two alleles may be the same or different on the two chromosomes.

The term “polymorphism”, as used herein, also is intended to refer to one of a group of possible DNA codings (e.g., variant sequences) occupying a particular position (locus) on a chromosome, but the term “polymorphism” is used when the range of possible variant sequences differ by only one or a small number of different nucleotides, whereas the term “allele” is used more broadly, encompassing situations in which the range of possible variant sequences differ more significantly, such as across the length of the protein coding region. For example, a “single nucleotide polymorphism” is intended to refer to one of a group of possible variants at a particular position (locus) on a chromosome, wherein the variants differ only at a single nucleotide position. Thus, single nucleotide polymorphisms of a gene represent different allelic variants of a gene. The term “restriction fragment length polymorphism” is intended to refer to a polymorphism within a gene (e.g., single nucleotide or several nucleotides) that creates or eliminates a site(s) for a restriction enzyme(s), as compared to the wild-type (i.e., most prevalent form) of the gene, such that digestion of the polymorphic form of the gene by the enzyme produces a DNA fragment(s) of a different length as compared to digestion of the wild-type form with the same enzyme.

The term “HLA-Cw6 allele”, as used herein, is intended to refer to a particular allele (Cw6) that can be present at the HLA-C major histocompatibility class I locus on the short arm of chromosome 6 in humans. The HLA-C locus can be occupied by one of at least 25 different Cw alleles, which have been identified based on sequence analysis (Zemmour, J. and Parham, P. (1993) Immunogenet. 37:239-250). The presence of the HLA-Cw6 allele at the HLA-C locus can be identified, for example, by the presence of a particular PCR-restriction fragment length polymorphism (see Tatari, Z. et al. (1995) Proc. Natl. Acad Sci. USA 92:8803-8807).

The term “TNFα 238 polymorphism”, as used herein, is intended to refer to a single nucleotide polymorphism at position −238 (upstream of the first exon) of the human tumor necrosis factor alpha (TNFα) gene, in which the nucleotide at position −238 is either an A or a G. This polymorphism is described further in D′Alfonso, S. et al (1994) Immunogenet. 39:150-154.

The term “TNFα 308 polymorphism”, as used herein, is intended to refer to a single nucleotide polymorphism at position −308 (upstream of the first exon) of the human tumor necrosis factor alpha (TNFα) gene, in which the nucleotide at position -308 is either an A or a G. This polymorphism, and detection thereof by NcoI restriction digestion of a PCR product, is described further in Wilson, A. G. et al. (1992) Hum. Mol Genet. 1:353.

The term “biologic therapy” is intended to refer to treatment of a subject with a biologic agent.

The term “biologic agent” as used herein is intended to encompass agents including proteins, antibodies, antibody fragments, fusion proteins, cytokines and similar naturally-occurring molecules, and/or recombinant and/or engineered forms thereof, that are produced in isolated form ex vivo such that they may be administered to a subject for therapeutic purposes.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

The terms “monoclonal antibody” or “monoclonal antibody composition” as used herein refer to a preparation of antibody molecules of single molecular composition. A monoclonal antibody composition displays a single binding specificity and affinity for a particular epitope.

The terms “chimeric antibody” or “chimeric monoclonal antibody” are intended to refer to antibodies in which the variable region sequences are derived from one species and the constant region sequences are derived from another species, such as an antibody in which the variable region sequences are derived from a mouse antibody and the constant region sequences are derived from a human antibody. Such “chimeric antibodies” can be prepared by standard recombinant technology well established in the art. For example, a nucleic acid encoding a V_{H} region from a mouse antibody can be operatively linked to a nucleic acid encoding the heavy chain constant regions from a human antibody and, likewise, a nucleic acid encoding a V_{L} region from a mouse antibody can be operatively linked to a nucleic acid encoding the light chain constant region from a human antibody.

The terms “humanized antibody” or “humanized monoclonal antibody” are intended to refer to antibodies in which CDR sequences derived from the germline of a non-human mammalian species, such as a mouse, have been grafted onto human framework sequences. Additional framework region modifications may be made within the human framework sequences. Such “humanized antibodies” can be prepared by standard recombinant technology well established in the art. For example, nucleic acids encoding the CDR1, CD2 and CDR3 regions from a V_{H} region of a mouse antibody can be operatively linked to nucleic acids encoding the FR1, FR2, FR3 and FR4 regions of a human V_{H} region, and the entire “CDR-grafted” V_{H} region can be operatively linked to nucleic acid encoding the heavy chain constant regions from a human antibody. Likewise, nucleic acids encoding the CDR1, CD2 and CDR3 regions from a V_{L} region of a mouse antibody can be operatively linked to nucleic acids encoding the FR1, FR2, FR3 and FR4 regions of a human V_{L} region, and the entire “CDR-grafted” V_{L} region can be operatively linked to nucleic acid encoding the light chain constant region from a human antibody.

The term “human antibody”, as used herein, is intended to refer to antibodies having variable regions in which both the framework and CDR regions are derived from human germline immunoglobulin sequences. Furthermore, if the antibody contains a constant region, the constant region also is derived from human germline immunoglobulin sequences. Human antibodies may include amino acid residues not encoded by human germline immunoglobulin sequences (e.g., mutations introduced by random or site-specific mutagenesis in vitro or by somatic mutation in vivo).

The term “human monoclonal antibody” refers to antibodies displaying a single binding specificity which have variable regions in which both the framework and CDR regions are derived from human germline immunoglobulin sequences. Human monoclonal antibodies can be produced by a hybridoma which includes a B cell obtained from a transgenic nonhuman animal, e.g., a transgenic mouse, having a genome comprising a human heavy chain transgene and a light chain transgene fused to an immortalized cell. The term “human monoclonal antibody”, as used herein, also includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. Such recombinant human antibodies, however, can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The terms “Ig fusion protein” and “Fc fusion protein” are intended to refer to a recombinant, composite protein comprising a polypeptide of interest operatively linked to a constant region portion of immunoglobulin, typically the hinge, CH₂ and CH₃ domains of heavy chain constant region, more typically the human IgG1 hinge, CH₂ and CH₃ domains. The polypeptide of interest operatively linked to the Fc portion can be, for example, a full-length protein or only a portion of a full-length protein, such as one or more extracellular domains of a protein, e.g., one or more extracellular domains of a cell-surface protein. Such “Ig fusion proteins” can be prepared by standard recombinant technology well established in the art. For example, a nucleic acid encoding the polypeptide of interest can be operatively linked to a nucleic acid encoding the hinge, CH₂ and CH₃ domains of a heavy chain constant region.

The term “immune response modifier” is intended to refer to a biologic agent that, directly or indirectly, has an effect on immune responses in a subject, typically through a direct or indirect effect on T cells and/or B cells in the subject.

The term “TNFα inhibitor” is intended to refer to a biologic agent that, directly or indirectly, inhibits TNFα activity, such as by inhibiting interaction of TNFα with a cell surface receptor for TNFα, inhibiting TNFα protein production, inhibiting TNFα gene expression, inhibiting TNFα secretion from cells or any other means resulting in decreased TNFα activity in a subject.

The term “LFA3 modulator” is intended to refer to a biologic agent that, directly or indirectly, modulates activity of LFA3 or a biochemical pathway mediated via LFA3, such as by modulating the interaction of LFA3 with CD2 on T cells, modulating the CD2 activation pathway in CD2⁺ T cells, modulating LFA3 and/or CD2 protein production, modulating LFA3 and/or CD2 gene expression, or any other means resulting in modulation of T cell responses via modulation of the LFA3/CD2 interaction pathway.

The term “CD11a modulator” is intended to refer to a biologic agent that, directly or indirectly, modulates activity of CD11a or a biochemical pathway mediated via CD11a, such as modulating a CD11a activation pathway in CD11a⁺ T cells, modulating CD11a protein production, modulating CD11a gene expression, or any other means resulting in modulation of T cell responses via modulation of CD11a activity.

As used herein, the term “subject” includes humans, and non-human animals amenable to biologic agent therapy, e.g., preferably mammals, such as non-human primates, sheep, dogs, cats, horses and cows.

As used herein, the term “psoriasis” is intended to encompass a range of dermatological conditions characterized by red, scaly patches and plaques or lesions associated with excessive skin production and inflammation, including mild, moderate or severe plaque psoriasis (psoriasis vulgaris), chronic plaque psoriasis and psoriasis with or without psoriatic arthritis. The severity of psoriasis in a subject typically is assessed based on the body surface area involved, with mild being less than 5%, moderate being 5 to 10% and severe being greater than 10%. In clinical research studies, the PASI (psoriasis area severity index) score is used to quantitate disease severity.

As used herein, the term “psoriasis subject” is intended to refer to a subject (e.g., human patient) suffering from psoriasis.

As used herein, the term “treatment regimen” is intended to refer to one or more parameters selected for the treatment of a subject, e.g., with a biologic agent, which parameters can include, but are not necessarily limited to, the type of agent chosen for administration, the dosage, the formulation, the route of administration and the frequency of administration.

Various aspects of the invention are described in further detail in the following subsections.

### Prediction of Responsiveness to Biologic Therapy for Psoriasis

In one aspect, the invention pertains to a method for predicting responsiveness to a biologic agent in a subject having psoriasis. Typically, the method comprises assaying the subject for the presence or absence of an allele or polymorphism predictive of responsiveness to biologic therapy in psoriasis, and predicting responsiveness of the subject to the biologic agent based on presence or absence of the allele or polymorphism in the subject.

Psoriasis has long been recognized as a disease that often clusters in families, suggesting a genetic basis for the disease, although Mendelian inheritance of the disease is not followed. Extensive studies have been performed to identify susceptibility genes for psoriasis, and a number of candidate susceptibility genes have been identified (reviewed in, for example, Ahmeen, M. (2003) Pharmacogenomics 4:297-308). The presence of the HLA-Cw6 allele has been found to be one of the strongest and most consistent indicators of susceptibility to psoriasis in many ethnically and geographically diverse groups (reviewed in, for example, Mallon, E. et al. (1999) J. Invest. Dermatol. 113:693-695). Furthermore, the TNFα 238 and TNFα 308 polymorphisms have shown an association with psoriasis, with the frequency of the TNFα 238 G/A genotype being increased in psoriasis patients as compared to control subjects and the frequency of the TNFα 308 G/A genotype being decreased in psoriasis patients as compared to control subjects (see e.g., Arias, A. I. et al. (1997) Exp. Clin. Immunogenet. 14:118-122; Hohler, T. et al. (1997) J. Invest. Dermatol. 109:562-565; Reich, K. et al. (1999) J. Invest. Dermatol. 113:214-220; Hohler, T. et al. (2002) Ann. Rheum. Dis. 61:213-218; Reich, K. et al (2002) J. Invest. Dermatol. 118:155-163; Mossner, R. et al. (2005) J. Invest. Dermatol. 124:282-2840).

Biologic therapy has begun to be used in psoriasis patients, although without any prior determination being made as to whether such therapy is likely to be effective in a particular patient. A comparison of the efficacy of certain biologic agents in psoriasis patients has been reported (see Dubertret, L. et al. (2006) Br. J. Dermatol. 155:170-181), summarized below in Table 1:

TABLE 1
Efficacy of PASI 75 for Biologic Therapies

| | | | | Percent |
|---|---|---|---|---|
| Generic Name | Brand Name | Dose | | Responders |
| Alefacept | AMEVIVE ™ | i.m. 15 | mg/wk | 21% |
| Efalizumab | RAPTIVA ™ | 1 | mg/kg/wk | 31% |
| Etanercept | ENBREL ™ | 25 | mg BIW | 34% |
| Etanercept | ENBREL ™ | 50 | mg BIW | 49% |
| Infliximab | REMICADE ™ | 5 | mg/kg | 80% |

To date, response to biologics has been based on clinical observations as no genetic markers have been reported to predict response to treatment. However, as discussed in further detail in the Examples, certain genetic markers have now been discovered, and are described herein, that allow for predicting responsiveness to a biologic agent in a subject having psoriasis, such that one can assay a subject for the presence or absence of an allele or polymorphism predictive of responsiveness to biologic therapy in psoriasis and, based on the presence or absence of the allele or polymorphism in the subject, one can predict responsiveness of the subject to the biologic agent, e.g., before initiation of treatment with the agent.

In one embodiment, the allele or polymorphism predictive of responsiveness to biologic therapy in psoriasis is an HLA-Cw6 allele. For example, presence of the HLA-Cw6 allele can be used to predict responsiveness of the subject to biologic therapy. Additionally or alternatively absence of the HLA-Cw6 allele can be used to predict lack of, or reduced, responsiveness of the subject to biologic therapy.

In another embodiment, the allele or polymorphism predictive of responsiveness to biologic therapy in psoriasis is a tumor necrosis factor alpha (TNFα) 238 polymorphism. For example, in one embodiment, presence of the G/A genotype at the TNFα 238 polymorphism site can be used predict responsiveness of the subject to biologic therapy. Additionally or alternatively, in this embodiment, absence of the G/A genotype and/or presence of the G/G genotype at the TNFα 238 polymorphism site can be used to predict lack of, or reduced, responsiveness of the subject to biologic therapy.

In another embodiment, presence of the G/G genotype at the TNFα 238 polymorphism site can be used predict responsiveness of the subject to biologic therapy. Additionally or alternatively, in this embodiment, absence of the G/G genotype and/or presence of the G/A genotype at the TNFα 238 polymorphism site can be used to predict lack of, or reduced, responsiveness of the subject to biologic therapy.

In another embodiment, the allele or polymorphism predictive of responsiveness to biologic therapy in psoriasis is a tumor necrosis factor alpha (TNFα) 308 polymorphism. For example, in one embodiment, presence of the G/G genotype at the TNFα 308 polymorphism site can be used predict responsiveness of the subject to biologic therapy. Additionally or alternatively, in this embodiment, absence of the G/G genotype and/or presence of the G/A genotype at the TNFα 308 polymorphism site can be used to predict lack of, or reduced, responsiveness of the subject to biologic therapy.

In another embodiment, presence of the G/A genotype at the TNFα 308 polymorphism site can be used predict responsiveness of the subject to biologic therapy. Additionally or alternatively, in this embodiment, absence of the G/A genotype and/or presence of the G/G genotype at the TNFα 308 polymorphism site can be used to predict lack of, or reduced, responsiveness of the subject to biologic therapy.

In still other embodiments, the allele or polymorphism predictive of responsiveness to biologic therapy in psoriasis is an HLA allele selected from the group consisting of HLA-Cw2, HLA-Cw4, HLA-Cw5, HLA-Cw6, HLA-Cw1, HLA-Cw3, HLA-Cw7 and HLA-Cw8. In still other embodiments, the allele or polymorphism predictive of responsiveness to biologic therapy in psoriasis is an allele of a killer cell Ig-like receptor (KR), such as KIR 2DL1 or KIR 2DL2.

In the method of the invention for predicting responsiveness to a biologic agent in a subject having psoriasis, the presence or absence of an allele or polymorphism predictive of responsiveness to biologic therapy in psoriasis can be assayed in the subject using techniques well-established in the art. In a preferred embodiment, the presence or absence of the allele or polymorphism in the subject is assayed by obtaining a genomic DNA sample from the subject and detecting the presence or absence of the allele or polymorphism in the genomic DNA sample (i.e., the allele or polymorphism is detected at the DNA level by examining genomic DNA from the subject) (discussed further below). Additionally or alternatively, in certain situations it may be possible to assay for the presence or absence of the allele or polymorphism at the protein level, using a detection reagent that detects the protein product encoded by the allele or polymorphic variant. For example, if an antibody reagent is available that binds specifically to the allele or polymorphic variant to be detected, and not to other alleles or polymorphic variants encoded by the gene under examination, then such an antibody reagent can be used to detect the presence or absence of the allele or polymorphism in a cellular sample from the subject.

Preferably, the allele or polymorphism in the subject is assayed by obtaining a genomic DNA sample from the subject and detecting the presence or absence of the allele or polymorphism in the genomic DNA sample. For example, a sample of peripheral blood can be obtained from the subject and genomic DNA can be isolated therefrom using standard technologies available in the art (e.g., the Puregene™ genomic DNA isolation kit, commercially available from Gentra Systems, Minneapolis, Minn.). Once the genomic DNA is obtained it can be analyzed for the presence or absence of the allele or polymorphism of interest using a detection approach designed to detect the allele or polymorphism of interest, such as detection of a restriction fragment length polymorphism that distinguishes the allele or polymorphism of interest from other variants of the gene.

For example, an assay for detecting the HLA-Cw6 allele, using a polymerase chain reaction-restriction fragment length polymorphism (PCR-RFLP), has been described in the art (Tatari, Z. et al. (1995) Proc. Natl. Acad. Sci. USA 92:8803-8807) and can be applied to the analysis of a genomic DNA sample from a psoriasis subject to determine the presence or absence of the HLA-Cw6 allele in the subject (described in further detail in Example 1).

Also for example, an assay for detecting the TNFα 238 and TNFα 308 polymorphisms, using photo-cleavable mass spectrometry tags (CMSTs) conjugated to oligonucleotide probes, has been described in the art (Kokoris, M. et al. (2000) Mol. Diagn. 5:329-340) and can be applied to the analysis of a genomic DNA sample from a psoriasis subject to determine the genotype of the TNFα 238 and TNFα 308 polymorphisms in the subject (described in further detail in Example 2).

The specific methods described above for detecting the HLA-Cw6 allele, the TNFα 238 polymorphism and/or the TNFα 308 polymorphism are representative examples only and are not intended to be limiting. Other suitable approaches for genotyping are known in the art. For example, for typing large numbers of single nucleotide polymorphisms (SNPs) rapidly, efficiently and accurately, the Invader™ technology can be used (described further in Mein, C. et al. (2000) Genome Research 10:330-343). Alternatively, SNPs can be evaluated using an amplification refractory mutation system-polymerase chain reaction (ARMS-PCR) technology such as described in Perrey, C. et al. (1999) Transplant Immunol. 7:127-128. Dinucleotide repeat polymorphisms can be evaluated by, for example, the method described in Polymeropoulos, M. H. et al. (1991) Nucl. Acids Res. 19:4018. It will be readily understood by the ordinarily skilled artisan that essentially any technical means established in the art for detecting alleles or polymorphisms can be adapted to the alleles and polymorphisms discussed herein and applied in the methods of the current invention for predicting responsiveness to a biologic agent in a subject having psoriasis.

Selection and Use of Treatment Regimens with Biologic Agents

Given the observation that the presence or absence of particular alleles or polymorphisms in a psoriasis subject influences the responsiveness of the subject to biologic therapy, one can select an appropriate treatment regimen for the subject based on the presence or absence of particular alleles or polymorphisms in the subject. Accordingly, in one embodiment, the above-described method for predicting the responsiveness to a biologic agent in a subject having psoriasis further comprises selecting a treatment regimen with the biologic agent based upon presence or absence of the allele or polymorphism in the subject. In another aspect, the method still further comprises administering the biologic agent to the subject according to the treatment regimen such that psoriasis is inhibited in the subject.

In another embodiment, the invention provides a method for selecting a treatment regimen for therapy with a biologic agent in a subject having psoriasis, the method comprising:

assaying the subject for presence or absence of at least one allele or polymorphism predictive of responsiveness to a biologic agent for treatment of psoriasis; and

selecting a treatment regimen with a biologic agent based upon presence or absence of the allele or polymorphism in the subject.

In yet another embodiment, the invention provides a method of treating a subject having psoriasis with a biologic agent, the method comprising:

assaying the subject for presence or absence of at least one allele or polymorphism predictive of responsiveness to a biologic agent for treatment of psoriasis;

selecting a treatment regimen with a biologic agent based upon presence or absence of the allele or polymorphism in the subject; and administering the biologic agent according to the treatment regimen such that the subject is treated for psoriasis.

The treatment regimen that is selected typically includes at least one of the following parameters and more typically includes many or all of the following parameters: the type of agent chosen for administration, the dosage, the formulation, the route of administration and/or the frequency of administration.

Preferred biologic agents include TNFα inhibitors, including but not limited to, infliximab (REMICADE™, a chimeric anti-TNFα antibody), etanercept (ENBREL™, a TNFR-Ig fusion protein) and adalimumab (HUMIRA™, a human anti-TNFα antibody), LFA3 modulators, including but not limited to alefacept (AMEVIVE™, an LFA3-Ig fusion protein) and/or CD11a modulators, including but not limited to efalizumab (RAPTIVA™, a humanized anti-CD11a mAb). Other examples of TNFα inhibitors include certolizumab pegol (CIMZIA™, a pegylated Fab′ fragment of a humanized anti-TNFα antibody), golimumab (a human anti-TNFα mAb) and onercept (a recombinant human soluble p55 TNFα binding protein).

Other suitable biologic agents include immune response modifiers in general. A large number of biologic agents that are immune response modifiers are currently under examination for the treatment of psoriasis, psoriatic arthritis or other types of autoimmune disorders, which biologic agents may be suitable for use in the current invention.

Non-limiting examples of such immune response modifiers that are chimeric monoclonal antibodies include: denoliximab (anti-CD4), galiximab (anti-CD80) and rituximab (RITUXAN™; anti-CD20).

Non-limiting examples of such immune response modifiers that are humanized monoclonal antibodies include: ABX-RB2 (anti-CD147), Antova (anti-CD40L), CDP850 (anti-E selectin), daclizumab (ZENAPAX™; anti-CD25), eculizumab (anti-C5), KM3 (anti-ICAM3), K20 (anti-CD29), MEDI-507 (anti-CD22), natalizumab (TYSABRI™; anti-integrin (alpha 4) VLA4) and visilizumab (NUVION™; anti-CD3).

Non-limiting examples of such immune response modifiers that are human monoclonal antibodies include: ABX-IL-8 (anti-IL-8), CNTO 1275 (anti-IL12/IL23), HuMax-IL-15 (anti-IL-15), MOR102 (anti-CD54) and HuMax-CD4 (anti-CD4).

Non-limiting examples of such immune response modifiers that are Ig fusion proteins include: abatacept (ORENCIA™; CTLA4-Ig) and rilonacept (IL1RAP-Ig).

Selection of the particular parameters of the treatment regimen can be based on known treatment parameters for the biologic agent previously established in the art. For example, a non-limiting example of a treatment regimen for alefacept is 15 mg/wk administered intramuscularly. A non-limiting example of a treatment regimen for efalizumab is 1 mg/kg/wk. A non-limiting example of a treatment regimen for etanercept is 25 or 50 mg twice a week (BIW). A non-limiting example of a treatment regimen for infliximab is 5 mg/kg. Other suitable treatment regimens for the biologic agents discussed herein will be readily apparent to the ordinarily skilled artisan based on prior studies of preferred administration parameters for the biologic agent.

For administration to a subject, a biologic agent typically is formulated into a pharmaceutical composition containing the biologic agent and a pharmaceutically acceptable carrier. Therapeutic compositions typically must be sterile and stable under the conditions of manufacture and storage. Pharmaceutical compositions also can be administered in combination therapy, i.e., combined with other agents, such as other biologic agents and/or other therapeutic agents, such as traditional therapeutic agents for the treatment of psoriasis).

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

The pharmaceutical compositions may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

A biologic agent of the present invention can be administered via one or more routes of administration using one or more of a variety of methods known in the art. As will be appreciated by the skilled artisan, the route and/or mode of administration will vary depending upon the desired results. A preferred route of administration, particularly for antibody agents, is by intravenous injection or infusion. Other preferred routes of administration include intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a biologic agent of the invention can be administered via a non-parenteral route, such as a topical, epidermal or mucosal route of administration, for example, intranasally, orally, vaginally, rectally, sublingually or topically.

In a preferred embodiment, the subject to be treated with the biologic agent is a human subject, more preferably a human subject that suffers from chronic plaque psoriasis. In one embodiment, the human subject suffers from chronic plaque psoriasis without psoriatic arthritis. In another embodiment, the human subject suffers from chronic plaque psoriasis with psoriatic arthritis.

### Kits of the Invention

In another aspect, the invention pertains to kits for carrying out the methods of the invention. For example, in one embodiment, the invention provides a kit for predicting responsiveness to a biologic agent in a subject having psoriasis. In one embodiment, the kit comprises:

a) means for detecting presence or absence in the subject of an allele or polymorphism predictive of responsiveness to biologic therapy in psoriasis; and

b) instructions for use of the kit to predicting responsiveness to a biologic agent in a subject having psoriasis.

In a preferred embodiment, the means for detecting presence or absence in the subject of an allele or polymorphism predictive of responsiveness to biologic therapy in psoriasis comprises a nucleic acid preparation sufficient to detect presence or absence of the allele or polymorphism in a genomic DNA sample from the subject. This nucleic acid preparation includes at least one, and may include more than one, nucleic acid probe or primer, the sequence(s) of which is designed such that the nucleic acid preparation can detect the presence or absence of the allele or polymorphism in a genomic DNA sample from the subject. A preferred nucleic acid preparation includes two or more PCR primers that allow for PCR amplification of a segment of the allele or polymorphic variant. Non-limiting examples of suitable PCR primers for amplification of a segment of the HLA-Cw6 allele, or the −238 polymorphic position of the TNFα gene, or the −308 polymorphic position of the TNFα gene, are described in further detail in Example 1.

Alternatively, the means for detecting presence or absence in the subject of an allele or polymorphism predictive of responsiveness to biologic therapy in psoriasis can comprise a reagent that detects the gene product of the allele or polymorphic variant of interest sufficient to distinguish it from other alleles or polymorphic variants in a sample from the subject. A non-limiting example of such a reagent is a monoclonal antibody specific for the allele or polymorphic variant.

The means for assaying the allele or polymorphism can also include, for example, buffers or other reagents for use in an assay for evaluating the allele or polymorphism. The instructions can be, for example, printed instructions for performing the assay for evaluating the allele or polymorphism.

In another embodiment, the kit can further comprise a biologic agent for treating psoriasis in the subject.

In a preferred embodiment, the kit includes means for detecting the presence or absence in the subject of an HLA-Cw6 allele as a predictor of responsiveness to biologic therapy in psoriasis. In this embodiment, the instructions can instruct the end user of the kit that the presence of the HLA-Cw6 allele predicts responsiveness of the subject to biologic therapy. Additionally or alternatively, the instructions can instruct the end user of the kit that the absence of the HLA-Cw6 allele predicts lack of, or reduced, responsiveness of the subject to biologic therapy.

In another embodiment, the kit includes means for detecting the presence or absence a G/A genotype and/or a G/G genotype at the TNFα 238 polymorphism as a predictor of responsiveness to biologic therapy in psoriasis. For example, in one embodiment, the instructions can instruct the end user of the kit that the presence of the G/A genotype at the TNFα 238 polymorphism predicts responsiveness of the subject to biologic therapy. Additionally or alternatively, in this embodiment, the instructions can instruct the end user of the kit that the absence of G/A genotype (or the presence of the G/G genotype) at the TNFα 238 polymorphism predicts lack of, or reduced, responsiveness of the subject to biologic therapy. In another embodiment, the instructions can instruct the end user of the kit that the presence of the G/G genotype at the TNFα 238 polymorphism predicts responsiveness of the subject to biologic therapy. Additionally or alternatively, in this embodiment, the instructions can instruct the end user of the kit that the absence of G/G genotype (or the presence of the G/A genotype) at the TNFα 238 polymorphism predicts lack of, or reduced, responsiveness of the subject to biologic therapy.

In another embodiment, the kit includes means for detecting the presence or absence a G/G genotype and/or a G/A genotype at the TNFα 308 polymorphism as a predictor of responsiveness to biologic therapy in psoriasis. For example, in one embodiment, the instructions can instruct the end user of the kit that the presence of the G/G genotype at the TNFα 308 polymorphism predicts responsiveness of the subject to biologic therapy. Additionally or alternatively, in this embodiment, the instructions can instruct the end user of the kit that the absence of G/G genotype (or the presence of the G/A genotype) at the TNFα 308 polymorphism predicts lack of, or reduced, responsiveness of the subject to biologic therapy. In another embodiment, the instructions can instruct the end user of the kit that the presence of the G/A genotype at the TNFα 308 polymorphism predicts responsiveness of the subject to biologic therapy. Additionally or alternatively, in this embodiment, the instructions can instruct the end user of the kit that the absence of G/A genotype (or the presence of the G/G genotype) at the TNFα 308 polymorphism predicts lack of, or reduced, responsiveness of the subject to biologic therapy.

In still other embodiments, the kit can include means for detecting the presence or absence of an HLA allele selected from the group consisting HLA-Cw2, HLA-Cw4, HLA-Cw5, HLA-Cw6, HLA-Cw1, HLA-Cw3, HLA-Cw7 and HLA-Cw8. In still other embodiments, the kit can include means for detecting the presence or absence of an allele of a killer cell Ig-like receptor (KIR), such as KIR 2DL1 or KIR 2DL2.

Preferred biologic agents for use with the kit include TNFα inhibitor, such as etanercept, infliximab and adalimumab, LFA-3 modulators, such as alefacept and/or CD11a modulators, such as efalizumab. Other suitable biologic agents include other immune response modifiers, non-limiting examples of which include the various biologic agents set forth above with respect selection and use of the treatment regimens.

Preferably, the kit is designed for use with a human subject, such as a human subject suffering from chronic plaque psoriasis. In one embodiment, the human subject suffers from chronic plaque psoriasis without psoriatic arthritis. In another embodiment, the human suffers from chronic plaque psoriasis with psoriatic arthritis.

### Databases and Computer Programs

In another aspect, the invention pertains to methods of building a database for use in selecting a psoriasis subject for treatment with a biologic agent, or for use in selecting or monitoring a treatment regimen in a psoriasis subject. The method includes receiving, in a computer system, genotypes, at one or more alleles or polymorphisms predictive of responsiveness to biologic agent treatment, from a plurality of subjects having psoriasis, and storing the genotype from each subject such that the genotype is associated with an identifier of the subject having that genotype, such as the name of the subject or a numerical identifier that is coded to an identity of the subject. The method of building the database can also include receiving, in the computer system, one or more treatment regimens for treatment of psoriasis in the subjects such that the treatment regimen(s) is associated with the genotype of the subject and the identifier of the subject. A user can enter the patient's genotype at the one or more alleles or polymorphisms, and optionally the patient's treatment regimen(s), into the computer system. Alternatively, the patient's genotype can be received directly from equipment used in determining the patient's genotype.

In another aspect, the invention pertains to a computer program product containing executable instructions that when executed cause a processor to perform operations relating to building a database for use in selecting a psoriasis subject for treatment with a biologic agent or for use in selecting or monitoring a treatment regimen for a psoriasis subject. The operations can include receiving, in a computer system, the genotype of a subject at one or more alleles or polymorphisms predictive of responsiveness to biologic therapy of psoriasis and storing the genotype such that the genotype is associated with an identifier of the subject, such as the name of the subject or a numerical identifier that is coded as corresponding to the subject. The operations can further include receiving, in the computer system, one or more treatment regimens for treatment of psoriasis in the subject such that the treatment regimen(s) is associated with the genotype of the subject and the identifier of the subject.

In yet another aspect, the invention pertains to a method of selecting a psoriasis subject for a treatment with a biologic agent using computer programs and databases of the invention. The method can include identifying, in a database comprising a plurality of psoriasis subjects, a subject whose database entry is associated with a genotype at one or more alleles or polymorphisms that is predictive of responsiveness to treatment with a biologic agent and selecting that subject for treatment with a biologic agent. The method can further comprise selecting a treatment regimen by identifying, in the database, a treatment regimen that has been associated with the genotype of the subject and with the identifier of the subject.

In yet another aspect, the invention pertains to a computer program product containing executable instructions that when executed cause a processor to perform operations that include identifying, in a database including a plurality of psoriasis subjects associated with genotypes, a subject that is associated with a genotype at one or more alleles or polymorphisms that is predictive of responsiveness to treatment with a biologic agent and outputting the identified subject as a subject to be treated with a biologic agent. The executable instructions when executed can further cause the processor to perform operations that include outputting a treatment regimen that is associated with the subject to be treated with the biologic agent.

Preferred genotypes at one or more alleles or polymorphisms that are predictive of responsiveness to treatment with a biologic agent, which can be received into the database, are as described herein, including the presence of the HLA-Cw6 allele, the presence of the G/A or G/G genotype at the TNFα 238 polymorphism and/or the presence of the G/G or G/A genotype at the TNFα 308 polymorphism. The genotypes at other alleles of polymorphisms that can be received by the database include genotypes of HLA alleles, such as those selected from the group consisting of HLA-Cw2, HLA-Cw4, HLA-Cw5, HLA-Cw6, HLA-Cw1, HLA-Cw3, HLA-Cw7 and HLA-Cw8, and genotypes of killer cell Ig-like receptor alleles, such as KIR 2DL1 or KIR 2DL2. Suitable biologic agents and treatment regimens for use with the databases and computer programs of the invention also are as described herein.

Computer systems and database software well established in the art can be adapted for use in the methods and computer program products of the invention for building and searching a database for use in selecting or monitoring a treatment regimen for a subject having psoriasis or for selecting a particular psoriasis subject for treatment with a biologic agent.

The present invention is further illustrated by the following examples which should not be construed as further limiting. The contents of all references, patents and published patent applications cited throughout this application are expressly incorporated herein by reference in their entirety.

### EXAMPLES

### Example 1

### HLA-Cw6 Allele as a Predictor of Responsiveness to Biologics

The Newfoundland and Labrador founder population was used to examine whether the presence or absence of the HLA-Cw6 allele correlated with responsiveness to biologic treatment in psoriasis patients. This Newfoundland and Labrador population represents a relatively homogeneous genetic population and has a high incidence of familial psoriasis (Nall, L. et al. (1999) Cutis 64:323-329).

Fifty-four psoriasis patients who had been treated with biologic agents were examined. The biologic agents used were alefacept (AMEVIVET™), efalizumab (RAPTIVA™), infliximab (REMICADE™), adalimumab (HUMIRA™), etanercept (ENBREL™) and onercept.

To perform genotyping of the patients, peripheral blood samples were collected by standard methods and processed using the Puregene™ genomic DNA isolation kit (Gentra Systems, Minneapolis, Minn.). The genomic DNA was assayed for the HLA-Cw6 allele, as follows.

HLA-Cw6 Genotyping

Genotypes for the HLA-Cw6 allele (homozygous Cw6, heterozygous Cw6 or homozygous not Cw6) were determined using a restriction enzyme assay previously described by Tatari et al. (Tatari, Z. et al. (1995) Proc. Natl. Acad. Sci. USA 92:8803-8807). Using this assay, a PCR product was produced from the genomic DNA as template using the primers 5′-GCTCCCACTCCATGAGGTATTTC-3′ (SEQ ID NO: 1) and 5′-GGTCAGTCTGTGCCTGGCGCTTGT-3′ (SEQ ID NO: 2), with an annealing temperature of 66° C. Five microliters of this product were digested with 5 Units of the restriction enzyme MspAI (New England Biolabs), by adding unpurified PCR product and enzyme for a total digestion time of at least 3.5 hours. This reaction was then electrophoresed on a 4% agarose gel. The presence of the Cw6 allele was indicated by the presence of two fragments following MspAI digestion, one 175 bp in length and the other 47 bp in length.

Results

The frequency of HLA-Cw6 allele in the examined patient population was 32%. The patient population was divided into responders (patients exhibiting responsiveness to treatment with the biologic agent) and non-responders (patients not exhibiting responsiveness to treatment with the biologic agent). Table 2 below summarizes the presence or absence of the HLA-Cw6 allele in the responder and non-responder populations.

TABLE 2
Presence or Absence of HLA-Cw6 in
Responders and Non-Responders

| | | | Non- | |
|---|---|---|---|---|
| Drug | Responders | HLA-Cw6 | Responders | HLA-Cw6 |
| AMEVIVE ™ | 4 | 2 pos | 2 | 1 - pos |
| | | 2 neg | | 1 - Neg |
| RAPTIVA ™ | 4 | 4 pos | 2 | 2 - neg |
| ENBREL ™ | 9 | 6 - pos | 4 | 3 - neg |
| | | 3 - neg | | 1 - pos |
| REMICADE ™ | 17 | 13 - pos | 3 | 2 - pos |
| | | 4 - neg | | 1 - neg |
| HUMIRA ™ | 7 | 3 - pos | 0 | |
| | | 4 - neg | | |
| Onercept | 0 | | 2 | 1 - pos |
| | | | | 1 - neg |

The results summarized in Table 2 indicate that presence of the HLA-Cw6 allele can be associated with responsiveness to biologic therapy, whereas absence of the HLA-Cw6 can be associated with lack of responsiveness to biologic therapy and, thus, that the presence or absence of the HLA-Cw6 allele can be used as a predictor of responsiveness to biologic therapy.

### Example 2

### TNFα Polymorphisms as Predictors of Responsiveness to Biologics

To examine whether the TNFα 238 and/or 308 polymorphisms are predictive of responsiveness to biologic agents in psoriasis, genotyping of psoriasis patients undergoing biologic therapy was performed at the TNFα 238 and 308 polymorphisms, as described in detail below. The frequency of the TNFα 238 G/G polymorphism in the examined patient population was 85% and the frequency of the TNFα 238 G/A polymorphism was 15%. The frequency of the TNFα 308 G/G polymorphism in the examined patient population was 68% and the frequency of the TNFα 238 G/A polymorphism was 32%.

To perform genotyping of the patients, peripheral blood samples were collected by standard methods and processed using the Puregene™ genomic DNA isolation kit (Gentra Systems, Minneapolis, Minn.). The TNFα 238 and TNFα 308 polymorphisms, both of which are polymorphisms with alleles of either A or G, were genotyped using a method developed by Qiagen Genomics Inc. (Kokoris, M. et al. (2000) Mol. Diagn. 5:329-340). This method utilizes photo-cleavable mass spectrometry tags (CMSTs) conjugated to oligonucleotide probes to accurately identify single nucleotide polymorphisms. In brief, asymmetrically amplified DNA was hybridized to CMST-conjugated oligonucleotide probes, unbound probe was washed away, and the purified material was injected into a single quadrapole mass spectrometer for analysis. As the probe-DNA duplex flows into the mass spectrometer, the CMSTs are cleaved from the oligonucleotide probes via a 254 nm low pressure mercury lamp. The mass spectrometer is able to quantitatively detect the amount of each CMST present for a given injection, which in turn directly correlates to the amount of each probe hybridized to the DNA. Allele calls are determined from the ratio of the extracted integrated ion current (EIIC) of the wild type (wt) CMST to the EIIC of the variant (vt) CMST. A wt to vt ratio of ≧2:1 indicates a homozygous wild type, a ratio of ≦0.5:1 indicates a homozygous variant and a ratio falling between 2:1 and 0.5:1 indicates a heterozygote.

In the case of the TNFα 238 and 308 polymorphisms, a single 328 base pair amplicon was used to perform the CMST allele typing. The forward PCR primer (5′-CCTGCATCCTGTCTGGAAGTT-3′; SEQ ID NO: 3) and the reverse PCR primer (5′-CTCATCTGGAGGAAGCGGTAG-3′; SEQ ID NO: 4) were purchased from Operon Technologies (Alameda, Calif.). The first stage PCR reactions were composed of 100 ng of genomic DNA, 0.5 μM forward and reverse primers, 10 mm Tris pH 8.3, 50 mM KCl, 1.5 mM MgCl₂, 0.2 mM each dNTP, 3 Units Taq DNA Polymerase (PCR reagents supplied by Boehringer Mannheim, Indianapolis, N. Mex.), and 660 ng TaqStart Antibody (Clontech, Palo Alto, Calif.). The second stage PCR reactions were identical to the first stage PCR reactions except that 10 μl of product from the first stage was used as the basis for amplification and the only primer added to the reaction was 0.5 μM of the reverse primer. Thermocycling conditions were was follows: 94° C. for 5 minutes initial denaturation; 40 cycles (first stage PCR) or 25 cycles (second stage PCR) of 94° C. for 30 seconds, 60° C. for 30 seconds, 72° C. for 1 minute; final extension at 72° C. for 5 minutes. A MJ Research 9600 thermocycler (MJ Research, Watertown, Mass.) was used for all PCR reactions. Products were visualized via a 2.0% agarose gel stained with ethidium bromide (Fisher Scientific, Fair Lawn, N.J.).

Sample analysis was performed as follows: The 328 base pair amplicon from the TNFα gene was amplified as indicated above. A competitive hybridization was then conducted in which 30 μl of the asymmetric amplicon was combined with 30 μl of a probe mixture containing 30 pmole of each of the four CMST probes, shown below in Table 3.

TABLE 3

CMST Probes Used for Genotyping TNFα Polymorphisms

| SNP | Probe | CMST | SEQUENCE | SEQ ID NO |
|---|---|---|---|---|
| TNFα 238 | wt | 419 or 479 | 5′-CCTCQGAATCGGAGCAQGGAG-3′ | 5 |
| TNFα 238 | vt | 423 or 439 | 5′-CCTCQGAATCAGAGCAQGGAG-3′ | 6 |
| TNFα 308 | wt | 495 or 527 | 5′-GAGGQGCATGGGGACGQGGTT-3′ | 7 |
| TNFα 308 | vt | 467 or 475 | 5′-GAGGQGCATGAGGACGQGGTT-3′ | 8 |

The hybridization buffer used consisted of 2 M guanidine isothiocyanate, 0.05% n-lauroyl-sarcosine, 5 mM Tris pH 7.5, and 2.5 mM EDTA (all purchased from Sigma, St. Louis, Mo.). The hybridization was for 15 minutes at 22° C. Following hybridization, unbound probe and hybridization buffer were removed from the CMST probe-DNA complex through use of Microcon-50 molecular weight cutoff filters (Amicon, Inc., Beverly, Mass.). The hybridization mixture was loaded into the Micron-50 then spun at 14,000×g for 6 minutes. The retained material was then washed twice with 400 μl of water (HPLC grade, Allied Signal Inc., Muskegon, N.J.) plus 0.1 μg/ml tRNA (Boehringer Mannheim, Indianapolis, Md.). Wash solution was removed at each step by centrifugation at 14,000×g for 7 minutes. The amplicon and associated hybridized CMST probe were recovered from the Micron-50 filter by adding 20 μl water+1.0 μg/ml tRNA then inverting and spinning the column at 1000×g for 2 minutes.

For mass spectrometry analysis, 18 μl of the sample was injected into the HP1100 MSD (Hewlett Packard, Palo Alto, Calif.) using selected ion monitoring for m/z 423/419 or 439/479 (TNFα 238) and 467/495 or 475/527 (TNFα 308). The HP1100 MSD consists of a HP1100 series LC/MS equipped with a vacuum degasser, binary pump, autosampler and diode array detector. The mass spectrometer was used with the APCI source option. HP LC/MSD Chemstation software operating on a HP vectra XA with Windows NT for workstations version 4.0 was used for system control, data acquisition and data analysis. The mobile phase flow stream into the MS consisted of 50% acetonitrile (AlliedSignal Inc., Muskegon, N.J.) in HPLC grade water (AlliedSignal Inc., Muskegon, N.J.) at a flow rate of 800 μl/minute. Samples were injected every minute with the gain set at 3. The photochemical cleavage device consisted of a 254 nm low pressure mercury lamp, a UV transparent reactor coil and a lamp holder (Aura Industries, Staten Island, N.J.). Peak integration parameters were set as follows: peak slope cutoff at 1000, peak width cutoff at 0.2, peak area cutoff at 500 and peak height cutoff at 500.

### Example 3

### Further Examination of HLA-Cw6 Allele as a Predictor of Responsiveness to Biologics

Additional psoriasis patients were examined to determine whether the presence or absence of the HLA-Cw6 allele correlated with responsiveness to biologic treatment. To perform the HLA-Cw6 genotyping, DNA was extracted from whole blood samples collected in sterile 10 mL collection tubes containing EDTA. Isolation of DNA was performed employing one of two methods, Promega Wizard Genomic DNA purification kit (product number PRA1620) or AutoGen Mini80 QuickGene DNA whole blood kit S (product number fk-dbs), both of which have been shown to yield highly pure DNA. DNA concentration was then measured by spectrophotometry and diluted to 30 ng/μL for polymerase chain reaction (PCR). PCR amplification was performed as indicated in the protocol that accompanied the kit (Olerup SSP™ HLA-Cw*06, product number 101.614-12). In brief, a cocktail was prepared containing DNA, PCR Master Mix complete with Taq, and water. This cocktail was then aliquoted into tubes that contained a dried primer solution consisting of a specific primer mix as well as a control primer pair. Sixteen PCR reactions with a volume of 10 μL were performed per sample. These tubes were then transferred to a thermal cycler and the cycling parameters were as follows:

| | | |
|---|---|---|
| 1 cycle | 94° C. | 2 min denaturation |
| 10 cycles | 94° C. | 10 sec denaturation |
| | 65° C. | 60 sec annealing and extension |
| 20 cycles | 94° C. | 10 sec denaturation |
| | 61° C. | 50 sec annealing |
| | 72° C. | 30 sec extension |

A 2% agarose gel was prepared using 0.5×TE buffer as indicated in the protocol. The PCR products were then loaded onto the gel along with a DNA size indicator. The gel was run in 0.5×TE buffer for 15-20 minutes at 8-10 volts/cm.

After electrophoresis was complete, the gel was transferred to a Bio Imager and then documented by photography under UV light. Presence and/or absence of each control band and specific PCR product was recorded for all 16 wells for each sample. Interpretation was performed by referencing the lot specific interpretation and specificity tables included with the kit.

To assess the responsiveness of psoriasis patients to treatment with a biologic agent, the PASI (psoriasis area severity index) score was used to quantitate disease severity. A “positive” response was considered to be a patient receiving a greater or equal to a PASI 75% improvement for the start of treatment (Baseline), whereas a “negative response” was considered to be a patient receiving less than 75% response.

Twenty-one patients who had been treated with the biologic agent efalizumab (RAPTIVA™) were examined. The data summarizing the presence or absence of the HLA-Cw6 allele and the responsiveness to RAPTIVA™ is shown below in Table 4:

TABLE 4
RAPTIVA ™ Responsiveness in HLA-Cw6
Positive and Negative Patients

| | |
|---|---|
| HLA-Cw6 | RAPTIVA ™ Responsiveness |

| Genotype | Negative | Positive | Total |
|---|---|---|---|
| Negative | 5 | 5 | 10 |
| Positive | 0 | 11 | 11 |
| Total | 5 | 16 | 21 |

The results showed that while 100% (11/11) of the HLA-Cw6 positive patients responded to the biologic agent, only 50% ( 5/10) of the HLA-Cw6 negative patients responded to biologic agent. The Fisher's Exact test (an analogue to the chi-squared test) was performed. P-value of the Fisher's Exact test was significant (P=0.012). Therefore, there was an association between presence of the HLA-Cw6 allele in the patients and responsiveness to the biologic agent RAPTIVA™.

What is claimed is:

1. A method for predicting responsiveness to a biologic agent in a subject having psoriasis, the method comprising: assaying the subject for presence or absence of an allele or polymorphism predictive of responsiveness to biologic therapy in psoriasis, and predicting responsiveness of the subject to the biologic agent based on presence or absence of the allele or polymorphism in the subject.

2. The method of claim 1, wherein the subject is assayed for presence or absence of the allele or polymorphism by obtaining a genomic DNA sample from the subject and detecting presence or absence of the allele or polymorphism in the genomic DNA sample.

3. The method of claim 1, which further comprises selecting a treatment regimen with the biologic agent based upon presence or absence of the allele or polymorphism in the subject.

4. The method of claim 2, which further comprises administering the biologic agent to the subject according to the treatment regimen such that psoriasis is inhibited in the subject.

5. The method of any one of claims 1-4, wherein the allele or polymorphism predictive of responsiveness to biologic therapy in psoriasis is an HLA-Cw6 allele.

6. The method of claim 5, wherein presence of the HLA-Cw6 allele predicts responsiveness of the subject to biologic therapy.

7. The method of claim 5, wherein absence of the HLA-Cw6 allele predicts lack of, or reduced, responsiveness of the subject to biologic therapy.

8. The method of any one of claims 1-4, wherein the allele or polymorphism predictive of responsiveness to biologic therapy in psoriasis is a tumor necrosis factor alpha (TNFα) 238 polymorphism.

9. The method of any one of claims 1-4, wherein the allele or polymorphism predictive of responsiveness to biologic therapy in psoriasis is a tumor necrosis factor alpha (TNFα) 308 polymorphism.

10. The method of any one of claims 1-9, wherein the biologic agent is a TNFα inhibitor.

11. The method of claim 10, wherein the TNFα inhibitor is selected from the group consisting of etanercept, infliximab and adalimumab.

12. The method of any one of claims 1-9, wherein the biologic agent is an immune response modifier.

13. The method of claim 12, wherein the biologic agent is an LFA-3 modulator.

14. The method of claim 13, wherein the LFA-3 modulator is alefacept.

15. The method of claim 12, wherein the biologic agent is a CD11a modulator.

16. The method of claim 15, wherein the CD11a modulator is efalizumab.

17. The method of any one of claims 1-16, wherein the subject is a human.

18. The method of claim 17, wherein the human subject suffers from chronic plaque psoriasis.

19. The method of claim 18, wherein the human subject suffers from chronic plaque psoriasis without psoriatic arthritis.

20. The method of claim 18, wherein the human subject suffers from chronic plaque psoriasis with psoriatic arthritis.

21. A kit for predicting responsiveness to a biologic agent in a subject having psoriasis, the kit comprising:
a) means for detecting presence or absence in the subject of an allele or polymorphism predictive of responsiveness to biologic therapy in psoriasis; and
b) instructions for use of the kit to predicting responsiveness to a biologic agent in a subject having psoriasis.

22. The kit of claim 21, wherein the means for detecting presence or absence in the subject of an allele or polymorphism predictive of responsiveness to biologic therapy in psoriasis comprises a nucleic acid preparation sufficient to detect presence or absence of the allele or polymorphism in a genomic DNA sample from the subject.

23. The kit of claim 21, which further comprises a biologic agent for treating psoriasis in the subject.

24. The kit of any one of claims 21-23, wherein the allele or polymorphism predictive of responsiveness to biologic therapy in psoriasis is an HLA-Cw6 allele.

25. The kit of claim 24, wherein presence of the HLA-Cw6 allele predicts responsiveness of the subject to biologic therapy.

26. The kit of claim 24, wherein absence of the HLA-Cw6 allele predicts lack of, or reduced, responsiveness of the subject to biologic therapy.

27. The kit of any one of claims 21-23, wherein the allele or polymorphism predictive of responsiveness to biologic therapy in psoriasis is a tumor necrosis factor alpha (TNFα) 238 polymorphism.

28. The kit of any one of claims 21-23, wherein the allele or polymorphism predictive of responsiveness to biologic therapy in psoriasis is a tumor necrosis factor alpha (TNFα) 308 polymorphism.

29. The kit of any one of claims 21-28, wherein the biologic agent is a TNFα inhibitor.

30. The kit of claim 29, wherein the TNFα inhibitor is selected from the group consisting of etanercept, infliximab and adalimumab.

31. The kit of any one of claims 21-28, wherein the biologic agent is an immune response modifier.

32. The kit of claim 31, wherein the biologic agent is an LFA-3 modulator.

33. The kit of claim 32, wherein the LFA-3 modulator is alefacept.

34. The kit of claim 31, wherein the biologic agent is a CD11a modulator.

35. The kit of claim 34, wherein the CD11a modulator is efalizumab.

36. The kit of any one of claims 21-35, wherein the subject is a human.

37. The kit of claim 36, wherein the human subject suffers from chronic plaque psoriasis.

38. The kit of claim 37, wherein the human subject suffers from chronic plaque psoriasis without psoriatic arthritis.

39. The kit of claim 37, wherein the human subject suffers from chronic plaque psoriasis with psoriatic arthritis.

40. A method of building a database for use in selecting a subject having psoriasis for treatment with a biologic agent, said method comprising: receiving, in a computer system, genotypes, at one or more alleles or polymorphisms predictive of responsiveness to biologic agent treatment, from a plurality of subjects having psoriasis; and storing the genotype from each subject such that the genotype is associated with an identifier of the subject.

41. The method of claim 40, wherein the identifier of the subject is a name of the subject.

42. The method of claim 40, wherein the identifier of the subject is a numerical identifier coded to an identity of the subject.

43. The method of claim 40, which further comprises receiving, in the computer system, one or more treatment regimens for treatment of psoriasis in a subject such that the treatment regimen is associated with the genotype of the subject and the identifier of the subject.

44. A computer program product containing executable instructions that when executed cause a processor to perform operations comprising: receiving, in a computer system, a genotype of a subject at one or more alleles or polymorphisms predictive of responsiveness to biologic therapy of psoriasis; and storing the genotype such that the genotype is associated with an identifier of the subject.

45. The computer program of claim 44, which further causes the processor to perform an operation comprising: receiving, in the computer system, a treatment regimen for treatment of psoriasis in the subject such that the treatment regimen is associated with the genotype of the subject and the identifier of the subject.

46. A method of selecting a psoriasis subject for a treatment with a biologic agent, the method comprising: identifying, in a database comprising a plurality of psoriasis subjects, a subject whose database entry is associated with a genotype at one or more alleles or polymorphisms that is predictive of responsiveness to treatment with a biologic agent; and selecting the subject for treatment with a biologic agent.

47. The method of claim 46, which further comprises selecting a treatment regimen by identifying, in the database, a treatment regimen that has been associated with the genotype of the subject and with an identifier of the subject.

48. A computer program product containing executable instructions that when executed cause a processor to perform operations comprising: identifying, in a database including a plurality of psoriasis subjects associated with genotypes, a subject that is associated with a genotype at one or more alleles or polymorphisms that is predictive of responsiveness to treatment with a biologic agent; and outputting the identified subject as a subject to be treated with a biologic agent.

49. The computer program of claim 48, which further cause the processor to perform an operation comprising outputting a treatment regimen that is associated with the subject to be treated with the biologic agent.


SEQUENCE LISTING (8 sequences)

SEQ ID NO: 1 - DNA, 23 bp, Artificial Sequence
  Description of Artificial Sequence Synthetic primer
gctcccactccatgaggtatttc

SEQ ID NO: 2 - DNA, 24 bp, Artificial Sequence
  Description of Artificial Sequence Synthetic primer
ggtcagtctgtgcctggcgcttgt

SEQ ID NO: 3 - DNA, 21 bp, Artificial Sequence
  Description of Artificial Sequence Synthetic primer
cctgcatcctgtctggaagtt

SEQ ID NO: 4 - DNA, 21 bp, Artificial Sequence
  Description of Artificial Sequence Synthetic primer
ctcatctggaggaagcggtag

SEQ ID NO: 5 - DNA, 21 bp, Artificial Sequence
  Description of Artificial Sequence Synthetic probe
cctcngaatcggagcanggag

SEQ ID NO: 6 - DNA, 21 bp, Artificial Sequence
  Description of Artificial Sequence Synthetic probe
cctcngaatcagagcanggag

SEQ ID NO: 7 - DNA, 21 bp, Artificial Sequence
  Description of Artificial Sequence Synthetic probe
gaggngcatggggacgnggtt

SEQ ID NO: 8 - DNA, 21 bp, Artificial Sequence
  Description of Artificial Sequence Synthetic probe
gaggngcatgaggacgnggtt